(19) United States
(12) Patent Application Publication
(10) Pub. No.: US 2024/0216310 A1
(43) Pub. Date: Jul. 4, 2024

(54) Rapid Onset Therapeutic Anti-Depression Ketosis Enabled by D-BetaHydroxyButyric Acid Systems, Methods and Compounds for Psilocybin co-administration

(71) Applicant: American Ketone Therapeutics LLC, Sheridan, WY (US)

(72) Inventor: Heidi H Hynes, Seattle, WA (US)

(21) Appl. No.: 18/398,952

(22) Filed: Dec. 28, 2023

Related U.S. Application Data
(60) Provisional application No. 63/477,780, filed on Dec. 29, 2022.

(51) Int. Cl. A61K 31/19; A61K 31/48; A61K 31/675; A61K 36/33; A61K 45/06; G01N 33/64

(52) U.S. Cl. CPC A61K 31/19; A61K 31/48; A61K 31/675; A61K 36/33; A61K 45/06; G01N 33/64

(57) ABSTRACT

The claimed uses of bioidentical free D-BetaHydroxyButyric Acid as a method (1303), system (1411) and optional co-adjuvant compound for pharmaceutical co-administration with Psilocybin compounds to treat Depression, Anxiety and Bipolar Disorder are novel, safe and not anticipated by the body of research spanning well over 75 years. Overall, no adverse findings have been reported within cell, animal and human safety studies at high dose levels with D-BetaHydroxyButyric acid which supports the safety and efficacy of the claimed uses at the anticipated usage levels for the disclosed applications.

### CROSS-REFERENCE TO RELATED APPLICATIONS

This patent application is related to and depends from previously filed provisional application Ser. No. 63/477,780 filed on Dec. 29, 2022.

### TECHNICAL FIELD

The claimed invention has applicability in pharmaceutical anti-depression medication. With greater particularity, the claimed invention is relevant to therapeutic interventions for Anti-Depression, Anti-Anxiety and mitigation of Bipolar Disorder. With still greater particularity, the claimed invention is directed at novel uses of the ketone body D-HydroxyButyric Acid for treatment of Depression, Anxiety and Bipolar Disorder together with and independently of pharmaceutical and naturally derived forms of Psilocybin along with related systems and methods.

### BACKGROUND OF THE INVENTION

Since the isolation of LSD by Albert Hoffmann in 1929, psychoactive substances have garnered substantial interest in the treatment of a wide variety of psychological disorders. Naturally occurring substances including psilocybin and peyote have had a prominent role in cultural ceremonies dating back hundreds of years. While there is a recent revitalization of psychoactive substances in the treatment of psychological disorders, the mechanisms of action are unknown and the potential for beneficial outcomes remains inconsistent.

Recent research has led to the development of new compounds derived from psilocybin, the active ingredient in magic mushrooms. Scientists have been focusing on creating novel derivatives of psilocin, the compound that psilocybin turns into in the body. These efforts have resulted in a pool of more than twenty-eight unique compounds, which exhibit diverse metabolic profiles and have shown potential in reducing the duration of psychedelic effects while maintaining therapeutic benefits.

Several major pharmaceutical companies and research institutions are involved in this area of research including GH Research: Located in Dublin, focusing on N,N-Dimethyltryptamine derivatives for treatment-resistant depression, Atai Life Sciences: Based in Berlin, serving as an incubator for companies developing psychedelic mental health treatments, Mind Medicine: Located in New York City, specializing in microdose and psychedelic-inspired medicines, Compass Pathways: Based in London, known for their Comp360 psilocybin therapy, Cybin: Located in Toronto, focusing on drug delivery of psychedelic compounds, Delix Therapeutics: Based in Boston, developing novel compounds based on psychedelics, Beckley Psytech: Located in Oxford, England, focusing on psychedelics for neurological and psychiatric disorders and Gilgamesh Pharmaceuticals: Based in New York City, developing novel psychedelic compounds using an artificial intelligence platform. Compass Pathways, for instance, has reported results from its Phase 2b clinical trial of Comp360, a patented polymorph of psilocybin. This study showed promising results for treatment-resistant depression. Eleusis is working on creating an infusion of psilocin, the active form of psilocybin, which could produce faster, less-variable results. Viridia Life Sciences, part of the Atai Life Sciences group, is developing new formulations of N,N-dimethyltryptamine (DMT) with the goal of creating treatments similar to Spravato, the ketamine nasal spray.

Overall, the focus in this area of research is not just on the development of these compounds but also on modifying their properties to make them safer, faster-acting, and potentially non-hallucinogenic while retaining their therapeutic benefits. Psilocybin derivatives, in general, share a tryptamine core attached to an ethyl amino group. The chemical structure of psilocybin can be described by substituting the given R groups as follows:

$$R 3 = R 4 = R 6 = R 7 = R 8 = R 9 = Hydrogen atom ( H ) \\ R 5 = Phosphate ester group = {OPO ( OH )}_{2} \\ R 1 = R 2 = Methyl group = {CH}_{3}$$

Examples of psilocybin derivatives include Psilocin, Norpsilocin, Aeruginascin, Baeocystin, Norbaeocystin, Bufotenin, and Bufotenidine. Recent research has focused on creating novel prodrug derivatives (NPDs) of psilocin, with a library of twenty-eight unique compounds represented by nine distinct prodrug classes. These compounds are engineered to modify the kinetics of the acute psychedelic response, potentially reducing the duration of the psychedelic effects while maintaining therapeutic benefits. Psilocybin Dosing: A typical psilocybin dose refers to the amount of the substance that induces a psychedelic experience or ‘trip’. This experience is characterized by alterations in perception, mood, and cognitive processes. In terms of quantity, the dose can vary based on factors like the individual's body weight, tolerance, and the desired intensity of the experience. However, a common range for a psychedelic experience is typically between 10 to 30 milligrams of pure psilocybin, or about 1 to 3 grams of dried psilocybin mushrooms, though these values can vary. These doses are intended to produce a noticeable and substantial alteration in consciousness, often used in therapeutic settings for treating conditions like depression or PTSD, or in spiritual or personal growth contexts.

On Psilocybin Microdosing: Microdosing involves taking a very small fraction of what would be considered a psychedelic dose of psilocybin, with the intention of not causing any substantial alterations in consciousness or perception. In terms of quantity, a typical microdose is about 1/10th to 1/20th of a full dose, which translates to approximately 0.1 to 1 milligram of pure psilocybin, or roughly 0.05 to 0.2 grams of dried mushrooms. Again, these values are approximate and can vary. The goal of microdosing is to achieve sub-perceptual effects. Users report improved mood, creativity, focus, and energy, among other benefits, without experiencing a full-blown psychedelic trip. Microdosing is often done in a regimented manner, such as taking the dose every few days. The quantitative difference, therefore, lies in the intent and desired outcome of the dosing. A full dose aims for a profound alteration of the mental state, while a microdose seeks to subtly enhance certain cognitive or emotional functions without significant alteration of one's usual perception or consciousness.

### BRIEF SUMMARY OF THE INVENTION

The ketogenic diet and more recently administration of ketogenic compounds have had a similar long history with proven benefits for conditions such as epilepsy. Moreover, dietary interventions for ketogenic diet treatment of bipolar disorder are yielding compelling results. The administration of the natural form of D-BetaHydroxyButyric Acid as an exogenous compound has rapid onset and direct benefits to brain bioenergenics. Preliminary data indicates that individuals suffering from mild to moderate depression often benefit from administration of D-BetaHydroxyButyric Acid in the absence of a salt or ester for the direct reduction of anxiety and depression. The claimed form of D-BetaHydroxyButyric Acid is bioidentical to the natural form of ketone generated endogenously and chemically distinguishable from the artificial ‘ketone salt’ and synthetic ‘ketone ester’. This application discloses two primary embodiments for the treatment of psychiatric depressive disorders: the use of bioidentical or ‘free D-BetaHydroxyButyric Acid’ alone as well as in combination with Psilocybin derived pharmaceuticals.

The claimed invention combines these two physiological approaches for psychological intervention into a combinatorial delivery system for improved physiological outcomes in the treatment of psychological conditions. The exogenous ketone D-BHB Acid is either co-administered or preceding the psychoactive compound with the following expected benefits:
1) Psychoactive compounds including psilocybin, peyote and LSD (and derivatives thereof) induce an extremely subjective state in the individual. While this extremely subjective state may result in clinical psychological breakthroughs for the individual, the possibility remains for negative individual experiences. With the introduction of D-BHB Acid improving brain bioenergenics and reducing individual anxiety, it is logical that negative individual experiences will be reduced leading to a higher percentage of positive psychological breakthroughs.
2) The present form of D-BHB Acid is highly palatable which is in direct contrast to the moderately unpalatable ketone salts and severely unpalatable ketone ester compounds. As taste often has a direct impact on the psychological experience of the individual it is believed that the greatly improved palatability of D-BHB will avoid imparting a negative subjective experience to the individual undergoing psychoactive substance treatment.

The rationale for a bioidentical D-BHB therapeutic regime is primarily supported by the neuroinflammation reduction properties of bioidentical D-BHB, high ketone levels generated by the same coupled with high palatability without salt load or liver metabolism requirements. As the bioidentical ‘free’ form. Moreover, the bioidentical ‘free’ D-BHB form is immediately bioavailable for rapid metabolism which is materially distinct from the ‘ketone salt’ compounds (often D+L racemic) chemically joined to a salt or the synthetic ‘ketone ester’ compounds which are alcohol based and must be processed in the stomach and liver before releasing the ketone contained therein.

As for the combination of bioidentical free D-BHB with Psilocybin compounds and derivatives, while the neurological mechanisms of action of both Psilocybin and D-BHB remain unclear, the combinatorial benefits include the positive ‘whole brain energy’ of bioidentical free D-BHB leading to a more positive psychiatric outcome. Bioidentical free D-BHB is preferentially consumed in the brain over glucose which creates a more positive energy environment for Psilocybin derived treatment while reducing neurological inflammation. In the complex environment of the human brain, the minutiae of mechanisms need not be fully understood to yield the benefits of the claimed combinatorial invention.

### BRIEF DESCRIPTION OF THE SEVERAL VIEWS OF THE DRAWING(S)

The accompanying drawings are included to better illustrate exemplary embodiments of the claimed invention.

FIG. 1 is a structural illustration of endogenous ketones and the claimed bioidentical free D-BetaHydroxyButyric Acid.

FIG. 2 is a structural illustration of chemical structures of D-BHB, salt and ketone ester.

FIG. 3 is a graphical illustration of endogenous ketone levels by age.

FIG. 4 is a graphical illustration of endogenous ketone brain metabolism.

FIG. 5 is a graphical illustration of comparative exogenous ketone blood levels.

FIG. 6 is a graphical illustration of brain metabolism of ketones.

FIG. 7 is a graphical illustration of endogenous D-BHB dietary based depression improvements.

FIG. 8 is a graphical illustration of D-BHB exogenous ketone blood levels.

FIG. 9 is a graphical illustration of D-BHB exogenous ketone blood levels.

FIG. 10 is a graphical illustration of continuous monitoring of body glucose levels.

FIG. 11 is a graphical illustration of continuous monitoring of body ketone levels resulting in improved deep sleep time due to enhanced D-BHB levels according to the claimed invention.

FIG. 12 is a schematic illustration of engineered gut microorganism with Central Nervous System sensory gene circuitry and D-BHB delivery system

FIG. 13 is a schematic illustration of a preferred D-BHB depression management method with optional Psilocybin administration.

FIG. 14 is a schematic illustration of a preferred D-BHB depression management system with optional Psilocybin administration.

### DETAILED DESCRIPTION OF THE INVENTION

Introduction: Depression in its many forms is a wide ranging mental disorder ranging from moderate anxiety to crippling bipolar disorder. The claimed invention alleviates depression by way of two primary embodiments. The first primary embodiment addresses depression by reducing neuro-inflammation and increasing direct brain energy by exogenously administering bioidentical D-BetaHydroxyButyric Acid. The second primary embodiment improves the anti-depressive benefits of psilocybin derived ingredients by pre-treatment or co-administration of D-BetaHydroxyButyric Acid to optimize pharmaceutical applications of psilocybin derived compounds.

I. D-Beta HydroxyButyric Acid Compound Disclosure and Anti-Depression applications.

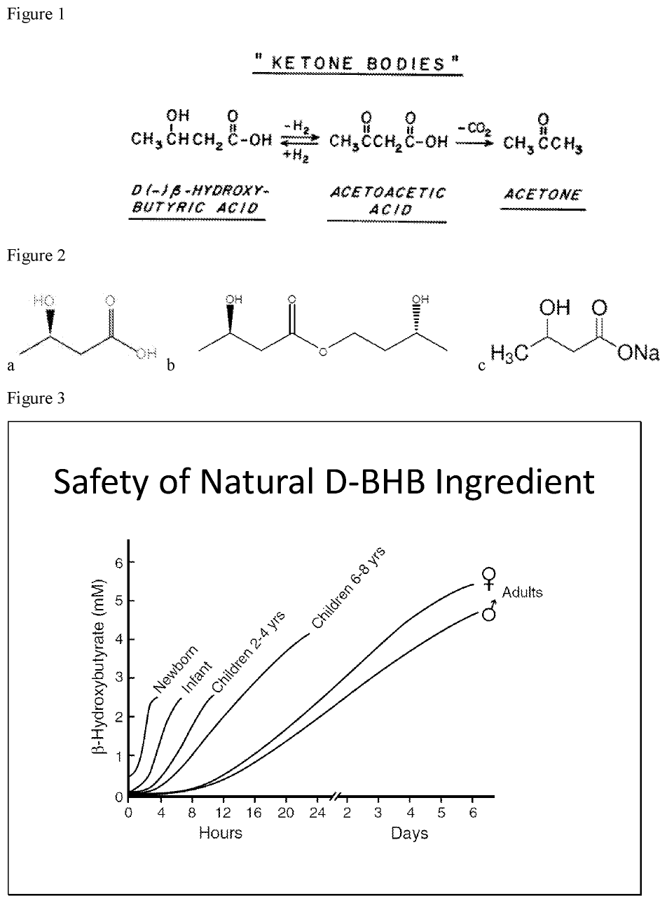

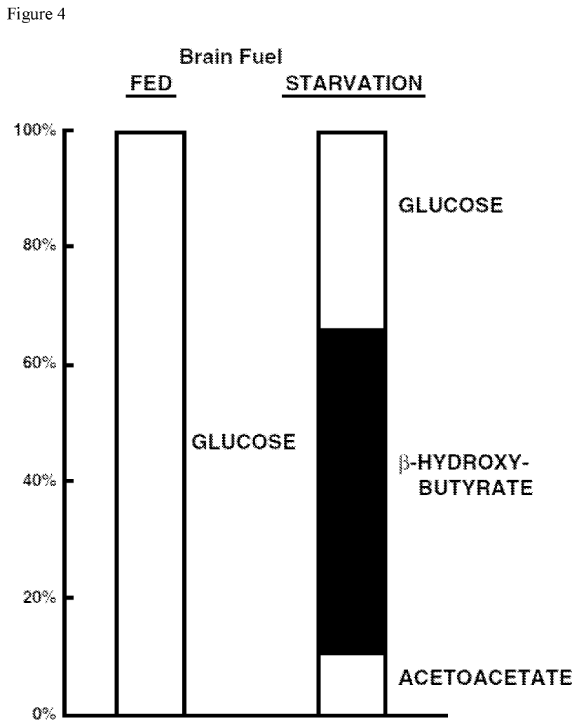

FIG. 1 is a structural illustration of endogenous ketones. The illustration of the endogenously produced ketone bodies is from FIG. 4 of the Harvard Medical School Professor/Dr. George F. Cahill, Jr.'s lecture entitled “Ketosis” given over fifty years ago. Dr. Cahill's foundational work provides clear and concise understanding of ketosis to aid in the understanding of the claimed invention. The FIG. 2 (a) chemical formula identifies the claimed bioidentical ‘free’ form of the exogenous D-Beta HydroxyButyric Acid administered in the absence of a chemical salt or ester and is the subject of ClinicalTrials.gov identifier NCT05584371. The claimed exogenously administered bioidentical free D-BetaHydroxyButyric Acid is chemically identical to the D-BetaHydroxyButyric Acid identified in FIG. 1. Greater detailed is hereby provided in the submitted BMJ Nutrition, Prevention & Health journal article. To be clear, this publication is the first reported clinical trial of bioidentical free D-BetaHydroxyButyric acid administered exogenously. The intervening fifty years between the Jeremiah Metzger Lecture given in Boston in 1972 and the present clinical trial and disclosure do not detail free bioidentical D-BetaHydroxyButyric Acid but instead disclose a wide variety of precursors, chemical combinations and not naturally occurring synthetic products which are not bioidentical in their administered form.

FIG. 2 is a structural illustration of chemical structures of bioidentical free BetaHydroxyButyric Acid represented in FIG. 2 (a), ketone ester FIG. 2 (b) and ketone salt FIG. 2 (c). Put simply, bioidentical free D-Beta HydroxyButyric Acid offers direct mitochondrial brain energy resulting in optimized therapeutic outcomes when administered in the absence of a chemical salt or ester configuration. To date, exogenous ketone supplementation has been attempted as an aide in following the ketogenic diet. Unfortunately, ketone salts result in an abnormally high salt intake rate and the newly created (R)-3-hydroxybutyl (R)-3-hydroxybutyrate (D-β-hydroxybutyrate ester) or ‘ketone ester’ is a never before seen in man synthetic creation with bioavailability and other long-term concerns.

FIG. 3 is a graphical illustration of endogenous ketone levels by age and FIG. 4 is a graphical illustration of endogenous ketone brain metabolism from Dr./Prof. Cahill's journal article “Fuel Metabolism in Starvation” provided by Information Disclosure Statement. FIG. 3 details the time to ketosis in healthy adults vs children measured in hours or days. FIG. 4 details the fact that ketones in general, and D-BetaHydroxyButyric Acid in particular is the preferred fuel in the human brain. The time to ketosis levels are relevant in the context of the instant application in that creating a rapid 1.5-2.5 mmol/L increase in ketone levels is simply not practical through fasting when rapidly needed in a depression onset or mental health episode prevention context.

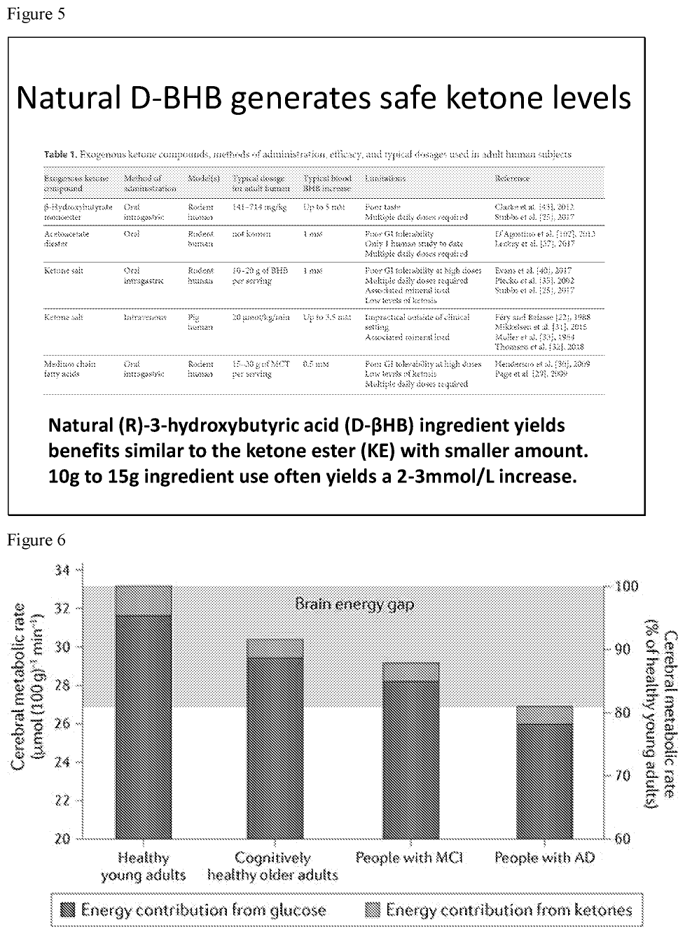

FIG. 5 is a graphical illustration of comparative exogenous ketone blood levels. FIG. 5 depicts Table 1 from “Exogenous Ketone Bodies as Promising Neuroprotective Agents for Developmental Brain Injury” and details the significant limitations among the variety of previously known exogenous salt and ester ketone compounds. Ketone salts are often racemic D+L mixtures which rarely provide greater than 1 mmol/L ketone increase along with substantial salt load rendering it unsuitable for long term daily administration. Similarly, while the synthetic ‘ketone ester’ does provide a higher ketone level response, its highly unpalatable taste renders it highly unsuitable for use in mental health applications. FIG. 5 does not include free D-BetaHydroxyButyric Acid due to the fact that ClinicalTrials.gov identifier NCT05584371 and the provided BMJ Nutrition, Prevention & Health journal article is the first instance of exogenous free D-BetaHydroxyButyric Acid journal publication. Put simply, there has been a longstanding need for a salt free, bioidentical solution to rapid ketosis without alcohol or need for further liver metabolism to rapidly increase ketones to a level relevant for mental health protection and recovery.

FIG. 6 is a graphical illustration of brain metabolism of ketones from Professor Stephen Cunnane's journal article “Brain energy rescue: an emerging therapeutic concept for neurodegenerative disorders of ageing” figure number 2. This figure details “Causes and consequences of the brain energy gap in neurodegenerative disorders . . . Glucose contributes to about 95% of total brain fuel supply in cognitively healthy young adults, and ketones supply the remaining 5%. In cognitively healthy older adults, brain glucose uptake is decreased by about 9%, in people with mild cognitive impairment (MCI) it is decreased by about 12% and in people with mild-to-moderate AD it is decreased by about 18%. The magnitude of the brain energy gap is the difference in total brain fuel uptake (glucose and ketones combined) between healthy young adults and people with mild-to-moderate AD; that is, the therapeutic target for brain energy rescue in MCI and AD. The brain energy gap has not been rigorously quantified in neurodegenerative disorders of ageing other than AD.” Applicants respectfully submits that Professor Cunnane's ‘brain energy gap’ model for mild cognitive impairment may be a useful framework and helpful analogy when addressing causes of depression disorders and a similar gap spectrum may exist when considering the differences between healthy individuals and individuals suffering from mild to substantial depression disorder. As supplementation with bioidentical free D-BetaHydroxyButyric Acid is direct mitochondrial brain energy it is the most suitable compound for restoration and closure of the ‘brain energy gap’ in depressive disorder individuals. As an optional anticipated embodiment, utilization of exogenous free D-BetaHydroxyButyric Acid to compensate for the brain energy gap deficiency will yield higher positive outcomes when utilizing Psilocybin derivatives for depressive disorders.

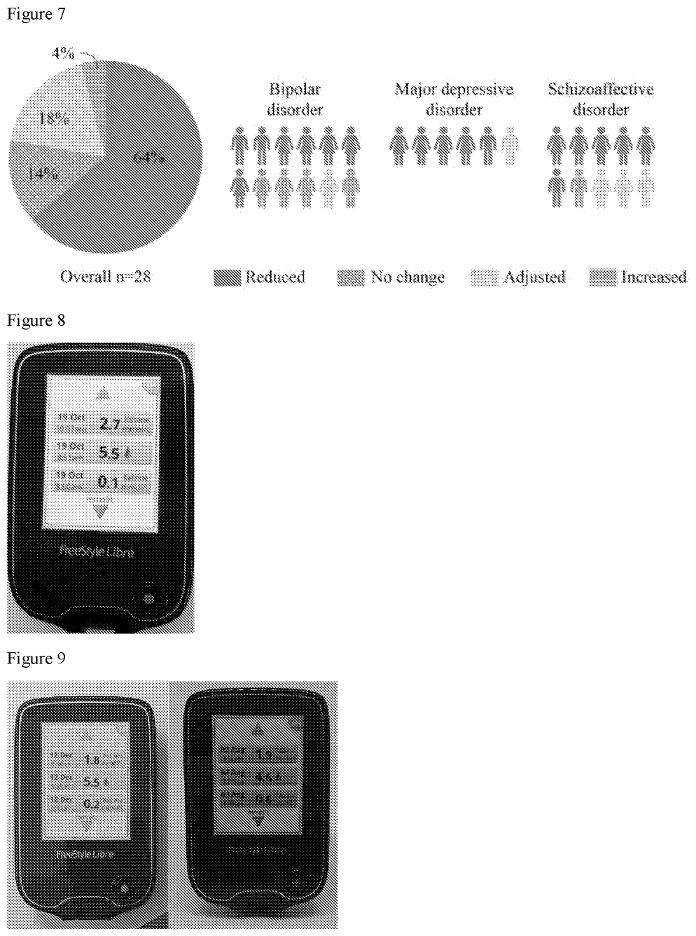

FIG. 7 is a graphical illustration of endogenous D-BHB dietary based depression improvements. In the recent Frontiers in Psychiatry article “The Ketogenic Diet for Refractory Mental Illness: A Retrospective Analysis of 31 Inpatients” the mental health benefits of the ketogenic diet summarized in the article's FIG. 2. While the benefits of the ketogenic diet in reducing the effects of bipolar disorder, major depressive disorder and schizoaffective disorder are laudable, controlling diet to increase ketone levels to therapeutic levels requires very strict regulation and control of carbohydrates and sugar along with high fat intake. Owing to the difficult dietary regimen, attempts to follow the ketogenic diet more often than not fail. Consequently, there is a long felt need and compelling benefit to utilizing bioidentical free D-BetaHydroxyButyric acid to rapidly increase ketone levels to mitigate (and potentially entirely avoid) psychiatric episodes related to depressive disorder.

FIG. 8 is a graphical illustration of D-BHB exogenous ketone blood levels. FIG. 8 details rapid ketosis confirmed by Abbott blood test strip after 15 g D-BHB with palatability agent administered by oral drink. Blood ketone levels document rise from 0.1 mmol/L to 2.7 mmol/L. By supplementing an individual with exogenous ketone preparations incorporating the natural D-Beta-HydroxyButyric Acid (D-BHB) optionally combined with a palatability agent, internal body ketone levels rapidly rise when directly compared with non-supplementing individuals. The ketone body D-BHB is first and foremost an energy source, which is directly and preferentially metabolized by the brain. D-BHB is also active as a signaling mechanism and neuro-inflammation reduction agent, resulting in broad and beneficial mental health related benefits.

FIG. 9 is a graphical illustration of D-BHB exogenous ketone blood levels. FIG. 9 details rapid ketosis within 45 minutes confirmed by Abbott blood test strip after 10 g D-BHB with palatability agent administered by oral drink. Blood ketone levels document rise from 0.2 mmol/L to 1.8 mmol/L and 0.6 mmol/L to 1.9 mmol/L in a variety of test subjects. FIG. 9 reinforces the fact that supplementation by utilizing free D only BetaHydroxyButyric Acid without ketone salt or ketone ester is a reliable, reproducible method and system for rapid ketone level increase for mental health protection and maintenance. While amounts of free D only BetaHydroxyButyric Acid taken for mental health anti-depression effects may vary, it is an anticipated preferred embodiment that levels are between 5 and 30 grams of D only BetaHydroxyButyric Acid taken as needed for optimum anti-depression effects. Alternate embodiments may include lower or higher amounts, however, experimental evidence has confirmed optimal administration in this range.

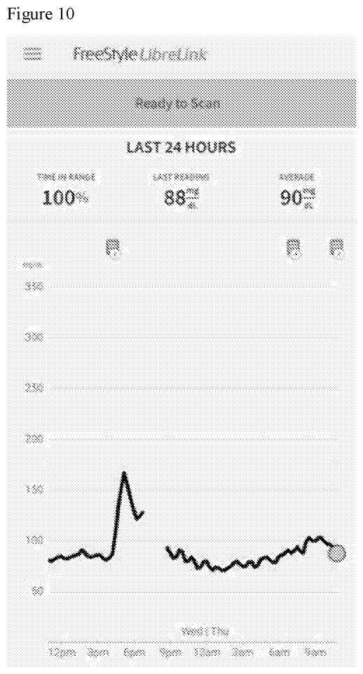

FIG. 10 is a graphical illustration of continuous monitoring of body glucose levels. FIG. 10 depicts continuous glucose monitoring levels with carbohydrate challenge documented by Abbot Libre Continuous Glucose Monitoring (CGM) device. In this illustrative embodiment, glucose levels from a 50 g carbohydrate ingestion are depicted unaided by free D-BetaHydroxyButyric Acid on the left and repeated the following day with two 50 g carbohydrate ingestions accompanied by 10 g free D-BetaHydroxyButyric Acid administered 45 minutes before the carbohydrate insult. The data is noteworthy in that while the equivalent amount of identical (nacho chip platter) carbohydrates were consumed, the rapid glucose spike depicted in the unsupplemented ingestion is not found with 10 g D-BetaHydroxyButyric Acid accompaniment. This data is relevant in the mental health context owing to the fact that certain individuals suffering from depression often report higher susceptibility due to low ketones and high glucose. As free D-BetaHydroxyButyric Acid can lower glucose while rapidly increasing ketone levels, it is believed that obtaining high ketones while lowering glucose will yield additional anti-depression benefits to high glucose sensitive individuals suffering from depression.

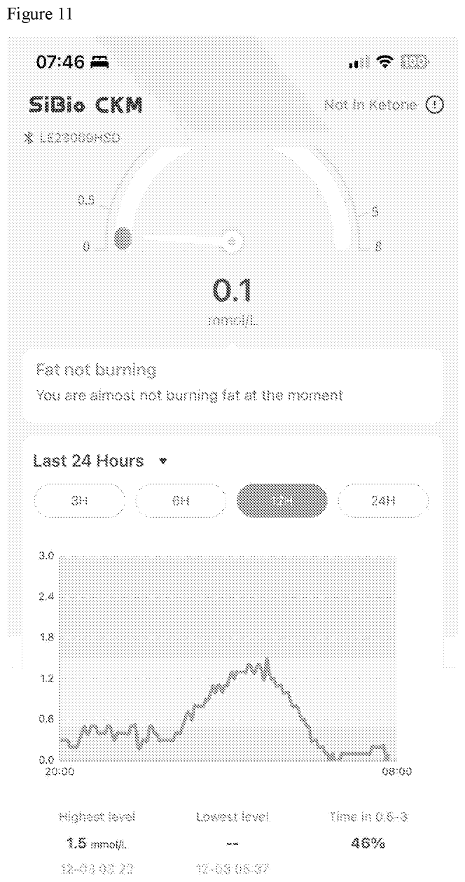

FIG. 11 is a graphical illustration of continuous monitoring of body ketone levels resulting in improved deep sleep time due to enhanced D-BHB levels according to the claimed invention. It is noteworthy that consumption of even small amounts of free D-BetaHydroxyButyric Acid can give rise to therapeutic ketosis levels as measured by Continuous Ketone Meter (CKM). In this representative example, 5 grams of free D-BetaHydroxyButyric Acid consumed 45 minutes prior to bedtime resulted in a 1.5 mmol/L increase can also enhance the quantity of deep sleep obtained. While free D-BetaHydroxyButyric Acid is not a sedative, even small amounts can greatly improve deep sleep quantity and quality. It is an intended and foreseeable embodiment application to increase deep sleep with free D-BetaHydroxyButyric Acid with the aim of improving mental health and anti-depression resiliency.

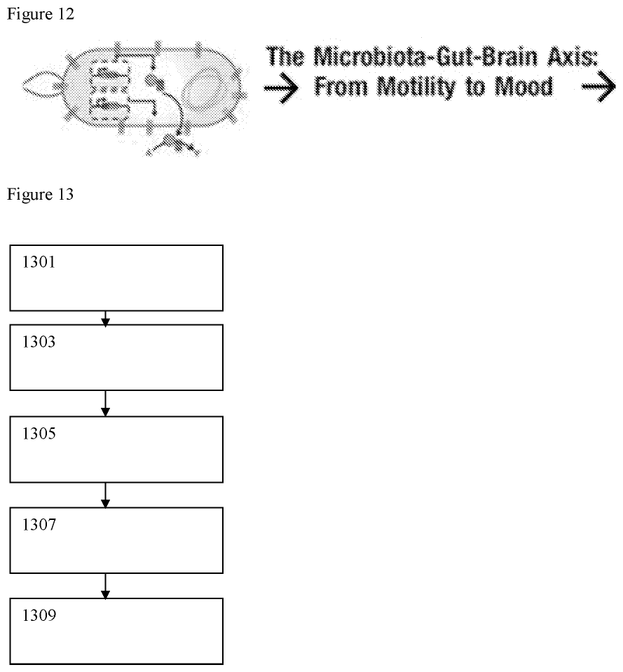

FIG. 12 is a schematic illustration of engineered gut microorganism with Central Nervous System sensory gene circuitry and D-BHB delivery system. The novel anti-depression benefits of exogenous D-Beta HydroxyButyric Acid administration arise from exogenous bolus administration in chemical form which may be thought of as intermittent and readily augmented by subsequent administration. As a foreseeable embodiment, a ‘sustained release’ format can be obtained through the use of an engineered ‘gut microorganism’ probiotic format. This system provides sustained and controllable administration of free D-BetaHydroxyButyric Acid by way of in body production from an engineered probiotic genetic expression vehicle capable of sensing increased need for D-BetaHydroxyButyric Acid for mental health applications along the gut-brain axis.

### II. D-Beta HydroxyButyric Acid Method & System Disclosure and Applications for Psilocybin Co-Administration

FIG. 13 is a schematic illustration of a preferred D-BHB depression management method with optional Psilocybin administration.

In a preferred embodiment, a method of depression symptom management is obtained through the steps of:
Assessing (1301) ketone levels and depressive symptoms,
Administering (1303) a therapeutic amount of free D-BetaHydroxyButyric acid,
Optionally dosing (1305) a therapeutic amount of Psilocybin derived substance,
Evaluating (1307) the depressive symptoms of a subject in need, and
Readministering (1309) free D-BetaHydroxyButyric acid alone or in combination with optionally redosing together with a therapeutic amount of Psilocybin derived material. Readily foreseeable embodiments of free D-BetaHydroxyButyric acid administration include administration between 5 grams and 30 grams of free D-BetaHydroxyButyric acid. Additionally, readily foreseeable embodiments of optional Psilocybin dosing and microdosing include administration between 10 to 30 milligrams of pure psilocybin, or about 1 to 3 grams of dried psilocybin mushrooms for dosing or micro-dosing at about 1/10th to 1/20th of a full dose.

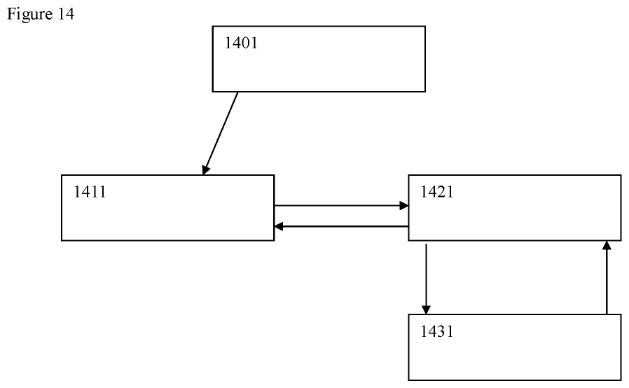

FIG. 14 is a schematic illustration of a preferred D-BHB depression management system with optional Psilocybin administration. In the illustrative embodiment, ketone level monitor (1421) determines the amount of free D-BetaHydroxyButyric Acid for a mental health subject in need thereof, optionally and additionally informed by glucose monitor (1433). Free D-BetaHydroxyButyric Acid therapeutic dose (1411) is administered alone or in conjunction with optional Psilocybin therapeutic dose (1401). Similar to the aforementioned method, readily foreseeable system embodiments of free D-BetaHydroxyButyric acid administration include administration between 5 grams and 30 grams of free D-BetaHydroxyButyric acid. Additionally, readily foreseeable system embodiments of optional Psilocybin dosing and microdosing include administration between 10 to 30 milligrams of pure psilocybin, or about 1 to 3 grams of dried psilocybin mushrooms for dosing or micro-dosing at about 1/10th to 1/20th of a full dose.

In the description, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments. In particular, free D-BetaHydroxyButyric Acid may be administered diluted or with a preferred palatability agent without detracting from the spirit or scope of the claimed invention.

Reference throughout this specification to “one embodiment”, “an embodiment”, “one example” or “an example” means that a particular feature, structure or characteristic described in connection with the embodiment or example is included in at least one embodiment of the present embodiments. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus. Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

I claim:

1. A method of depression symptom management comprising the steps of:
Assessing ketone levels and depressive symptoms,
Administering a therapeutic amount of free D-BetaHydroxyButyric acid,
Evaluating the depressive symptoms of a subject in need, and
Readministering free D-BetaHydroxyButyric acid until depressive symptoms are reduced.

2. The method of claim 1 wherein said Administering a therapeutic amount of free D-BetaHydroxyButyric Acid is provided between 5 grams and 30 grams of free D-BetaHydroxyButyric Acid.

3. The method of claim 1 additionally comprising dosing a therapeutic amount of Psilocybin derived substance as a co-adjuvant simultaneously delivered with Administering a therapeutic amount of Free D-BetaHydroxyButyric Acid.

4. The method of claim 1 additionally comprising dosing a therapeutic amount of Psilocybin derived substance as a co-adjuvant subsequently delivered more than 15 minutes after Administering a therapeutic amount of Free D-BetaHydroxyButyric Acid.

5. The method of claim 1 additionally comprising dosing a therapeutic amount of Psilocybin derived substance as a co-adjuvant subsequently delivered more than 15 minutes before Administering a therapeutic amount of Free D-BetaHydroxyButyric Acid.

6. The method of claim 3 additionally comprising Readministering free D-BetaHydroxyButyric Acid in combination with redosing with a therapeutic amount of Psilocybin derived material.

7. The method of claim 6 wherein said Psilocybin derived material is between 10 to 30 milligrams of pure psilocybin.

8. The method of claim 6 wherein said Psilocybin derived material is approximately 0.1 to 1 milligram of pure psilocybin.

9. A depression management system comprising a ketone level monitor which determines the amount of free D-BetaHydroxyButyric Acid for a mental health subject in need thereof, a glucose monitor and Free D-BetaHydroxyButyric Acid therapeutic dose.

10. The depression management system of claim 9 wherein the amount of free D-BetaHydroxyButyric Acid administered is between 5 grams and 30 grams of free D-BetaHydroxyButyric acid.

11. The depression management system of claim 9 additionally comprising a Psilocybin therapeutic dose.

12. The depression management system of claim 10 wherein the amount of Psilocybin therapeutic dose is between 10 to 30 milligrams of pure psilocybin.

13. The depression management system of claim 10 wherein the amount of Psilocybin therapeutic dose is approximately 0.1 to 1 milligram of pure psilocybin.

14. A therapeutic compound comprising D-BetaHydroxyButyric Acid administered in 5 g-30 g format in conjunction with a psychoactive substance selected from the group consisting of psilocybin, peyote and LSD administered in a psychologically therapeutic amount.

15. The therapeutic compound of claim 14 additionally comprising psilocybin derivatives Psilocin, Norpsilocin, Aeruginascin, Baeocystin, Norbaeocystin, Bufotenin, and Bufotenidine.

16. The therapeutic compound of claim 14 additionally comprising psilocybin novel prodrug derivatives (NPDs) of psilocin.
